(19) United States
(12) Patent Application Publication
(10) Pub. No.: US 2021/0330191 A1
(43) Pub. Date: Oct. 28, 2021

(54) Wearable Temperature Indicator

(71) Applicant: Fardad Zanjani, Burlington Township, NJ (US)

(72) Inventor: Fardad Zanjani, Burlington Township, NJ (US)

(21) Appl. No.: 17/241,679

(22) Filed: Apr. 27, 2021

Related U.S. Application Data
(60) Provisional application No. 63/016,006, filed on Apr. 27, 2020.

(51) Int. Cl. A61B 5/01; A61B 5/00; A42B 1/012

(52) U.S. Cl. CPC A61B 5/01; A61B 5/6803; A61B 2562/0276; A42B 1/012; A61B 5/743

(57) ABSTRACT

A wearable indicator device includes: a flexible sheet having a first surface with an adhesive covering at least a portion of the first surface and a second surface configured to be positioned proximate to skin of a wearer of the wearable indicator device; and a visual temperature indicator. The visual temperature indicator is configured to be visible to bystander(s) when the wearable indicator device is being worn and changes in appearance when exposed to a temperature corresponding to a temperature exceeding a normal body temperature of the wearer to provide a visual indication when the wearer has an abnormal body temperature. The visual temperature indicator is connected to, embedded in, or integral with the flexible sheet, such that the flexible sheet is configured to hold the visual temperature indicator proximate to the skin of the wearer.

### CROSS REFERENCE TO RELATED APPLICATION

The present application claims the benefit of U.S. Provisional Patent Application No. 63/016,006, filed Apr. 27, 2020, the disclosure of which is hereby incorporated by reference in its entirety.

### BACKGROUND OF THE INVENTION

### Field of the Invention

This disclosure is directed to a wearable temperature indicator that can be mounted to or integral with a wearable item, such as a garment, hat, hairnet, facemask, headband, bracelet, patch, or bracelet, including a temperature sensitive material that provides a visual indicator, which can be viewed by others, showing when a wearer has an abnormally high body temperature.

### Description of Related Art

Abnormally high body temperature (e.g., a fever) is a symptom of a variety of medical conditions including infections (e.g., strep throat, flu, chickenpox, or pneumonia), rheumatoid arthritis, overexposure of skin to sunlight or sunburn, heat stroke, and/or dehydration. Significantly, fever is a common symptom of coronavirus infections, such as Coronavirus disease 2019 (COVID-19). In order to identify individuals with coronavirus disease or other medical conditions, commercial businesses, workplaces, transportation facilities, and other venues, which attract crowds of people, may periodically take individuals' temperatures to identify sick individuals. For example, shift workers may have their temperature taken at the start of a shift to ensure that they are healthy and will not infect others. Commercial businesses may use infrared thermometers or infrared cameras to scan groups of people entering the establishment to identify sick individuals.

However, there is a need for other types of indicators that show on a continuous basis when an individual does not have a fever or when the individual has a fever, so that appropriate action may be taken. Appropriate can include, for example, directing the individual to return home or to seek medical attention. The temperature-sensitive devices of the present disclosure are designed to provide a low-cost, easy-to-use alternative for identifying individuals who have a fever without the need to repeatedly take an individual's temperature using a handheld thermometer or using expensive infrared imaging devices.

### SUMMARY OF THE INVENTION

According to an aspect of the disclosure, a wearable indicator device includes: a flexible sheet having a first surface with an adhesive covering at least a portion of the first surface and a second surface configured to be positioned proximate to skin of a wearer of the wearable indicator device; and a visual temperature indicator. The visual temperature indicator is configured to be visible to bystander(s) when the wearable indicator device is being worn and changes in appearance when exposed to a temperature corresponding to a temperature exceeding a normal body temperature of the wearer to provide a visual indication when the wearer has an abnormal body temperature. The visual temperature indicator is connected to, embedded in, or integral with the flexible sheet, such that the flexible sheet is configured to hold the visual temperature indicator proximate to the skin of the wearer.

According to another aspect of the disclosure, a hairnet includes a wearable support structure including a fabric sheet configured to cover at least a portion of a wearer's head and/or hair and at least one elastic strap connected to the fabric sheet positioned to secure the fabric sheet to the wearer's head. The hairnet further includes a visual temperature indicator mounted to the wearable support structure configured to be visible to bystander(s) when the hairnet is being worn and which changes in appearance when exposed to a temperature corresponding to a temperature exceeding a normal body temperature of the wearer to provide a visual indication when the wearer has an abnormal body temperature. The wearable support structure is configured to hold the visual temperature indicator proximate to a forehead of the wearer.

According to another aspect of the disclosure, a method of assembling a hairnet includes a step of providing a hairnet with a fabric sheet configured to cover at least a portion of a wearer's head and/or hair and at least one elastic strap positioned to secure the fabric sheet to the wearer's head. The method further includes adhering a first surface of a wearable support structure of a wearable indicator device to the fabric sheet of the hairnet by pressing the indicator device against an underside of the fabric sheet such that an adhesive on the first surface of the wearable indicator device adheres to the fabric sheet of the hairnet. When the indicator device is adhered to the fabric sheet of the hairnet, a visual temperature indicator of the indicator device is positioned such that, when the hairnet is worn by the wearer, the visual temperature indicator is proximate to a forehead of the wearer.

Examples of the present invention will now be described in the following numbered clauses:

Clause 1: A wearable indicator device, comprising: a flexible sheet comprising a first surface comprising an adhesive covering at least a portion of the first surface and a second surface configured to be positioned proximate to skin of a wearer of the wearable indicator device; and a visual temperature indicator configured to be visible to bystander(s) when the wearable indicator device is being worn and which changes in appearance when exposed to a temperature corresponding to a temperature exceeding a normal body temperature of the wearer to provide a visual indication when the wearer has an abnormal body temperature, wherein the visual temperature indicator is connected to, embedded in, or integral with the flexible sheet, such that the flexible sheet is configured to hold the visual temperature indicator proximate to the skin of the wearer.

Clause 2: The indicator device of clause 1, wherein the visual temperature indicator comprises a temperature-sensitive material that undergoes a chemical reaction causing the material to change in appearance when exposed to the temperature corresponding to the temperature exceeding normal body temperature.

Clause 3: The indicator device of clause 2, wherein the temperature-sensitive material comprises at least one of a solution, film, coating, powder, or particles integral with or embedded in a polymeric substrate.

Clause 4: The indicator device of clause 2 or clause 3, wherein the temperature sensitive material has a first appearance when the temperature to which the temperature sensitive material is exposed corresponds to normal body temperature, and the temperature sensitive material has a second appearance when the temperature to which the temperature sensitive material is exposed corresponds to abnormal body temperature.

Clause 5: The indicator device of clause 4, wherein the temperature sensitive material is configured to transition between the first appearance and the second appearance multiple times as the temperature to which the temperature sensitive material is exposed changes between the temperature corresponding to normal body temperature and the temperature corresponding to abnormal body temperature.

Clause 6: The indicator device of clause 4, wherein the temperature sensitive material initially has the first appearance, transitions to the second appearance when the temperature to which the temperature sensitive material is exposed corresponds to abnormal body temperature, and is incapable of returning to the first appearance following the transition from the first appearance to the second appearance.

Clause 7: The indicator device of any of clauses 4 to 6, wherein the temperature sensitive material has the first appearance when at least a portion of the temperature sensitive material that can be seen by the bystander(s) comprises a first color, and the temperature sensitive material has the second appearance when the portion of the temperature sensitive material changes from the first color to a different second color.

Clause 8: The indicator device of clause 7, wherein the temperature sensitive material has a third appearance when the temperature to which the temperature sensitive material is exposed corresponds to the wearer having a body temperature significantly exceeding normal body temperature by more than about 3 degrees Fahrenheit.

Clause 9: The indicator device of any of clauses 1 to 8, wherein the visual temperature indicator changes in appearance when exposed to the temperature corresponding to the abnormal body temperature by displaying at least one of a word, phrase, or icon visible to the bystander(s) indicating to the bystander(s) that at least one of (i) the wearer has a fever, (ii) the wearer should seek medical attention, or (iii) the bystander(s) should remain socially distanced from the wearer.

Clause 10: The indicator device of any of clauses 1 to 9, wherein the visual temperature indicator has an inactive or pre-use appearance when exposed to temperatures significantly below normal body temperature to indicate that the visual temperature indicator is not in-use or is not correctly positioned proximate to the wearer's skin.

Clause 11: The indicator device of any of clauses 1 to 10, wherein the flexible sheet comprising multiple laminated layers and the visual temperature indicator comprises at least one temperature-sensitive layer laminated within the multiple layers of the wearable support structure and at least partially visible through the multiple layers of the wearable support structure.

Clause 12: The indicator device of clause 11, wherein the multiple laminated layers comprise flexible polymer layers comprising at least one of polyvinyl chloride, polyethylene, polyurethane, or latex.

Clause 13: The indicator device of any of clauses 1 to 12, wherein the visual temperature indicator comprises multiple portions configured to change color when exposed to different temperatures to provide an indication of a severity of the wearer's fever.

Clause 14: The indicator device of any of clauses 1 to 13, wherein the first surface of the flexible sheet is configured to be adhered to an underside of a head covering worn by the wearer, the indicator device further comprising a removable cover sheet over the adhesive configured to be removed from the device prior to adhering the device to the underside of the head covering.

Clause 15: The indicator device of any of clauses 1 to 14, wherein the first surface of the flexible sheet is configured to be adhered to an underside of a head covering worn by the wearer, the indicator device further comprising: a second adhesive covering at least a portion of the second surface of the flexible sheet so that the device can be adhered either to the underside of the head covering or directly to the wearer's skin; a first removable cover sheet over the adhesive on the first surface configured to be removed from the device when adhering the device to the underside of the head covering; and a second removable cover sheet over the second adhesive on the second surface configured to be removed from the device when adhering the device directly to the wearer's skin.

Clause 16: A hairnet comprising: a wearable support structure comprising a fabric sheet configured to cover at least a portion of a wearer's head and/or hair and at least one elastic strap connected to the fabric sheet positioned to secure the fabric sheet to the wearer's head; and a visual temperature indicator mounted to the wearable support structure configured to be visible to bystander(s) when the hairnet is being worn and which changes in appearance when exposed to a temperature corresponding to a temperature exceeding a normal body temperature of the wearer to provide a visual indication when the wearer has an abnormal body temperature, wherein the wearable support structure is configured to hold the visual temperature indicator proximate to a forehead of the wearer.

Clause 17: The hairnet of clause 16, further comprising a clip that attaches the visual temperature indicator to the wearable support structure.

Clause 18: The hairnet of clause 16 or clause 17, wherein the visual temperature indicator is removably connected to the wearable support structure by an adhesive.

Clause 19: A method of assembling a hairnet, the method comprising: providing a hairnet comprising a fabric sheet configured to cover at least a portion of a wearer's head and/or hair and at least one elastic strap positioned to secure the fabric sheet to the wearer's head; and adhering the first surface of the wearable support structure of the wearable indicator device of any of clauses 1 to 15 to the fabric sheet of the hairnet by pressing the indicator device against an underside of the fabric sheet such that the adhesive adheres to the fabric sheet of the hairnet, wherein, when the indicator device is adhered to the fabric sheet of the hairnet, the visual temperature indicator of the indicator device is positioned such that, when the hairnet is worn by the wearer, the visual temperature indicator is proximate to a forehead of the wearer.

Clause 20: The method of clause 19, wherein adhering the first surface of the wearable support structure to the fabric sheet comprises removing a cover sheet from the wearable indicator device to expose the adhesive and pressing the first surface and the adhesive of the wearable indicator device against the underside of the fabric sheet of the hairnet.

These and other features and characteristics of the present invention, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. As used in the specification and the claims, the singular form of “a”, “an”, and “the” include plural referents unless the context clearly dictates otherwise.

### BRIEF DESCRIPTION OF THE DRAWINGS

FIG. 1A is a schematic drawing of a top view of a wearable indicator device, according to an aspect of the disclosure;

FIG. 1B is a cross-sectional view of the wearable indicator device of FIG. 1A taken along line 1B-1B;

FIG. 1C is a schematic drawing of a top view of the wearable indicator device of FIG. 1A with the cover sheet of the device removed exposing the adhesive and the visible temperature indicator;

FIG. 2A is a schematic drawing of another example of a wearable indicator device;

FIG. 2B is a cross-sectional view of the wearable indicator device of FIG. 2A taken along line 2B-2B;

FIG. 3A is a front view of a hairnet with a wearable indicator device, according to an aspect of the disclosure;

FIG. 3B shows a hairnet and wearable indicator device being worn by a person where the indicator shows that the person has a normal body temperature;

FIG. 3C shows an individual wearing the hairnet, where the wearable indicator device shows that the individual has an abnormally high body temperature;

FIG. 4 shows an individual wearing a head covering and facemask with wearable indicator devices attached to the head covering and to the facemask, according to an aspect of the disclosure;

FIG. 5 shows an individual wearing a face shield including a wearable indicator device, according to an aspect of the disclosure;

FIGS. 6A and 6B show disposable bracelets including wearable indicator devices, according to aspects of the present disclosure;

FIG. 7 shows a reusable or washable bracelet including a wearable indicator device, according to an aspect of the disclosure;

FIG. 8 is a perspective view of a hat including a wearable indicator device, according to an aspect of the disclosure;

FIGS. 9A and 9B show chef's hats including a wearable indicator device, according to aspects of the disclosure;

FIG. 10A shows a headband worn by an individual including a wearable indicator device;

FIG. 10B is a perspective view of another example of a headband including a wearable indicator device;

FIG. 11A is a perspective view of a pair of safety glasses including a wearable indicator device showing that a wearer has a normal body temperature, according to an aspect of the disclosure;

FIG. 11B is a perspective view of the pair of safety glasses where the wearable indicator device shows that the wearer has an abnormally high body temperature;

FIG. 12A is a schematic drawing of another example of a temperature indicator, according to an aspect of the disclosure;

FIG. 12B is a schematic drawing showing the temperature indicator of FIG. 12A attached to a disposable bracelet; and

FIG. 12C is a schematic drawing showing the temperature indicator of FIG. 12A attached to a hat.

### DETAILED DESCRIPTION OF THE PRESENT INVENTION

For purposes of the description hereinafter, the terms “upper”, “lower”, “right”, “left”, “vertical”, “horizontal”, “top”, “bottom”, “lateral”, “longitudinal,” and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

With reference to the figures, a wearable indicator device 10, 110, 210 comprises a support structure 12, 112, 212 and a visual temperature indicator 14, 114, 214 that detects a temperature of the wearer's skin to determine whether the individual has a fever. The support structure 12, 112, 212 is configured to hold the visual temperature indicator 14, 114, 214 proximate to the wearer's skin. As used herein, the indicator 14, 114, 214 is “proximate to” the skin when it is close enough to the skin to detect a temperature of the skin with reasonable accuracy. For example, the device 10, 110, 210 can be positioned against the skin, such that at least a portion of the visual temperature indicator 14, 114, 214 directly contacts the wearer's skin. In other examples, there may be a gap or empty space between the visual temperature indicator 14, 114, 214 and the skin. In other examples, there may be another structure, such as a paper or plastic layer, film, or cover, between the visual temperature indicator 14, 114, 214 and the skin.

The wearable indicator device 10, 110, 210 is intended to be worn on a conspicuous location on the wearer's body, such as a forehead, face, chest, neck, arm, or wrist, so that other people (i.e., bystanders) can clearly see the visual temperature indicator 14, 114, 214. The wearable indicator device 10, 110, 210 provides on a continuous basis a visual confirmation for others that the wearer does not have a fever. Beneficially, this visual confirmation that the wearer does not have a fever is provided for as long as the device 10, 110, 210 is worn, such as for a worker's entire shift or for a customer's entire visit to a commercial establishment. Also, the wearable indicator device 10, 110, 210 shows when the wearer develops a fever so that appropriate action can be taken, such as instructing the wearer to return home or to seek medical attention.

In some examples, the wearable support structure 12, 112, 212 can comprise an article of clothing or garment which, desirably, is easily seen by others. The support structure 12, 112, 212 can also comprise a head covering, such as a hairnet or hat. In other examples, the support structure 12, 112, 212 comprises a facemask, headband, or bracelet. In some examples, the support structure 12, 112, 212 is intended to be used as a disposable, single use garment. Alternatively, the support structure 12, 112, 212 can be washable, so that it can be reused multiple times.

In some examples, the visual temperature indicator 14, 114, 214 comprises a temperature sensitive material configured to change in appearance when exposed to a temperature exceeding normal body temperature (e.g., 98.6° F.). For example, the temperature sensitive material may be formed or impregnated with a temperature-sensitive solution, film, coating, powder, or particles that undergoes a chemical reaction causing the material to change in appearance when exposed to a temperature that exceeds normal body temperature. In some examples, the temperature sensitive material comprises a standalone non-liquid temperature sensitive material, such as a powder or film embedded with the visual temperature indicator 14, 114, 214. In some examples, the temperature sensitive material is food safe (e.g., consumable, non-toxic, biodegradable, and/or biocompatible) so that the device 10, 110, 210 can be worn by food service providers, such as workers in a cafeteria and/or commercial kitchen.

In some examples, the temperature sensitive material may return to its initial appearance once the detected temperature returns to normal body temperature. In other examples, the temperature sensitive material is single-use, meaning that once it undergoes a change in appearance signifying that the wearer's temperature exceeds normal body temperature, the material does not return to its initial appearance.

As used herein, a “change in appearance” may be a color change (e.g., a change in color from green to red to show that the wearer has a fever). In other examples, a word, phrase, or icon may be revealed as the temperature change is detected. For example, the visual temperature indicator 14, 114, 214 may show a message (e.g., “Seek Medical Attention”) when the wearer's body temperature is above normal body temperature. Conversely, the visual temperature indicator 14, 114, 214 may show a message, such as “SAFE” or “OK”, when the wearer's body temperature is within a normal temperature range.

In some examples, the visual temperature indicator 14, 114, 214 only has two possible appearances or states (e.g., a first state when temperature is within a normal range and a second state when temperature is above a normal range). In other examples, the visual temperature indicator 14, 114, 214 may show more than two states. For example, the visual temperature indicator 14, 114, 214 may be green when the wearer's body temperature is normal (e.g., 98.6° F. or below). The visual temperature indicator 14, 114, 214 may be yellow or orange when the wearer has a mild fever (e.g., body temperature of from 98.6° F. to 100° F.). The visual temperature indicator may be red when the wearer has a more severe fever (e.g., body temperature of 3° F. above normal body temperature or a body temperature of above 100° F.). Additionally, the temperature indicator may display a different color if the temperature sensor 14, 114, 214 is inactive (i.e., is not positioned on a wearer) or is being worn improperly so that the temperature indicator 14, 114, 214 is not detecting changes in the wearer's skin temperature. This alerts bystanders that the indicator 14, 114, 214 is not actively sensing the wearer's body temperature and should not be relied upon to show whether the wearer has a fever. In other examples, the visual temperature indicator 14, 114, 214 may comprise a visual display that shows numeric values for temperature sensed by the temperature sensitive material, such as numeric values comprising one or two significant digits.

The visual temperature indicator 14, 114, 214 can be connected to, embedded in, or integral with the support structure 12, 112, 212 and/or with a garment worn by the wearer. Desirably, the support structure 12, 112, 212 holds the temperature sensitive material of the indicator 14, 114, 214 proximate to the wearer's skin so that accurate temperature readings can be collected. In some examples, the visual temperature indicator 14, 114, 214 is connected or secured to the support structure 12, 112, 212 by stitching, fasteners, or adhesives, as are known in the art. For example, the visual temperature indicator 14, 114, 214 may be connected to the support structure 12, 112, 212 by sewing or gluing. In other examples, the support structure 12, 112, 212 may comprise a pocket on a skin-facing side or surface of the support structure 12, 112, 212. The visual temperature indicator 14, 114, 214 may be sized to slide into the pocket so that one side of the indicator 14, 114, 214 is proximate to the wearer's skin and an opposing side of the indicator 14, 114, 214 can be seen by others.

In other examples, the visual temperature indicator 14, 114, 214 can be molded with or embedded in the support structure 12, 112, 212. For example, the temperature sensitive material of the visual temperature indicator 14, 114, 214 can be laminated in layers of the support structure 12, 112, 212 using conventional lamination processes, as are known in the art.

In another example, the support structure 12, 112, 212 comprises a flat sheet, similar in size to a BAND-AID® bandage, comprising opposing front and back sides. The visual temperature indicator 14, 114, 214 can be embedded in and/or connected to the support structure 12, 112, 212. Both the front and back sides of the flat sheet of the support structure 12, 112, 212 may comprise adhesive so that a user can adhere the visual temperature indicator 14, 114, 214 either directly to skin or to the underside of a head covering, such as a hat or hairnet.

In another example, the support structure 12, 112, 212 comprises a connector configured to be secured to the wearer's clothing or head covering. For example, the connector may comprise a foldable resilient clip operating in a fashion of, for example, a paper clip. The visual temperature indicator 14, 114, 214 can be secured to the connector or clip in a position where one side of the indicator 14, 114, 214 is proximate to the wearer's skin and another side of the indicator 14, 114, 214 is visible to others. For example, a foldable resilient clip may attach to a peripheral edge of the wearer's garment supporting the visual temperature indicator 14, 114, 214 over the peripheral edge of the garment, so that one side of the indicator 14, 114, 214 is proximate to the skin and an opposing side of the indicator 14, 114, 214 is visible to others.

### Wearable Indicator Device Examples

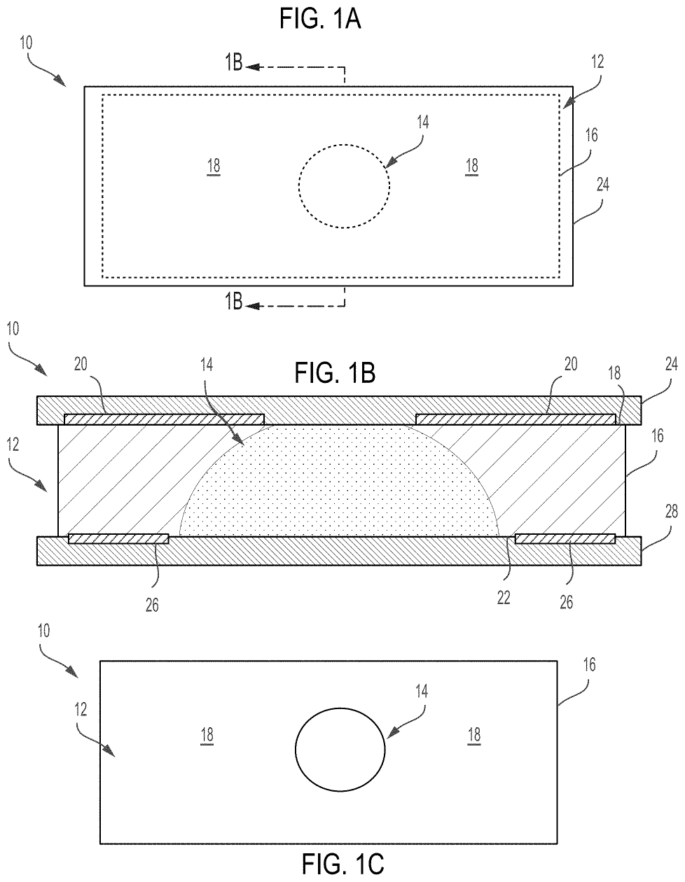

With reference to FIGS. 1A-1C, in some examples, the wearable indicator device 10 is configured to be worn by a wearer (i.e., a server worker, guest of a commercial establishment, or other individuals) to provide visual confirmation that the wearer does not have a fever. The indicator device 10 comprises a support structure 12 comprising an adhesive sheet 16 having a first surface 18 comprising an adhesive 20 covering at least a portion of the first surface 18 and a second surface 22 configured to be positioned proximate to skin of a wearer of the wearable indicator device 10. The adhesive sheet 16 can be formed from laminated or coated paper, woven fabrics, or from polymeric materials, such as polyvinyl chloride, polyethylene, polyurethane, or latex. The adhesive 20 can comprise an acrylate, methacrylate, epoxy diacrylate, or other adhesive materials commonly used for adhesive bandages, stickers, and similar disposable products. The adhesive 20 can cover the entire first surface 18 of the support structure 12 or only selected portions of the first surface 18. In either case, the adhesive 20 can be positioned to adhere the first surface 18 to an underside of a wearable item, such as a garment or head covering worn by the wearer, in a position which is easily visible to bystanders. When the wearable item is being worn, the second surface 22 of the indicator device 10 is positioned proximate to the wearer's skin.

The wearable indicator device 10 further comprises the visual temperature indicator 14, which is configured to be visible to bystander(s) when the wearable indicator device 10 is being worn and which changes in appearance when exposed to a temperature corresponding to a temperature exceeding a normal body temperature of the wearer to provide a visual indication when the wearer has an abnormal body temperature. As used herein, a “temperature corresponding to a temperature exceeding a normal body temperature” means a temperature sensed or detected by the visual temperature indicator 14 that indicates that the body temperature of the wearer exceeds normal body temperature (i.e., exceeds 98.6° F.). In some examples, the actual temperature sensed or detected by the visual temperature indicator may be the same as the actual body temperature (i.e., 98.6° F.) of the wearer, while in other examples, the temperature sensed or detected by the visual temperature indicator may be higher or lower than the actual body temperature of the wearer. A person skilled in the art can calibrate the visual temperature indicator 14 of the present disclosure to determine what temperature sensed or detected by the visual temperature indicator 14 corresponds to normal or abnormal body temperature of the wearer.

In some examples, the visual temperature indicator 14 can be reusable (i.e., capable of changing appearance multiple times when exposed to normal and abnormal body temperatures) or can be a single-use device which transitions to indicate abnormal body temperature and, once changed, does not return to an initial appearance.

The visual temperature indicator 14 can be connected to, embedded in, or integral with the support structure 12, such that the support structure 12 holds the visual temperature indicator 14 proximate to the wearer's skin. The indicator 14 is configured to be visible to bystander(s) when the wearable indicator device 10 is being worn by the wearer. The indicator 14 can comprise a solution, film, coating, powder, or particles integral with or embedded in a polymeric substrate that are temperature sensitive and which change in appearance when temperature changes occur. As shown in FIGS. 1A-1C, the indicator 14 has a circular cross section and is entirely enclosed by the wearable support structure 12. In other examples, the indicator 14 can be any convenient shape that is either fully or partially enclosed and supported by the support structure 12. In other examples, the indicator 14 can be a separate structure that is connected to a side or peripheral edge of the support structure 12 for maintaining positioning of the indicator 14 proximate to the wearer's skin. The indicator 14 changes in appearance when exposed to a temperature corresponding to a temperature exceeding a normal body temperature of the wearer to provide a visual indication when the wearer has an abnormal body temperature. For example, the visual temperature indicator 14 can comprise a temperature-sensitive material that undergoes a chemical reaction causing the material to change in appearance when exposed to the temperature corresponding to the temperature exceeding normal body temperature.

In some examples, the indicator device 10 further comprising a removable cover sheet 24 over the adhesive 20 and the first surface 18 of the adhesive sheet 16 configured to be removed from the device 10 prior to adhering the device 10 to the wearable item (i.e., a garment or head covering). For example, the first surface 18 and the adhesive 20 could be adhered to an underside of a head covering, such as a hairnet. The removable cover sheet 24 can comprise paper, plastics, or any other convenient material.

In some examples, the wearable indicator device 10 further comprises a second adhesive 26 covering at least a portion of the second surface 22 of the support structure 12 so that the device 10 can be adhered directly to the wearer's skin instead of to the wearable item. As with the adhesive 20 on the first surface 18, the adhesive 26 on the second surface 22 can cover the entire second surface 22 of the support structure 12 or only part of the second surface 22. The indicator device 10 can also comprise a second removable cover sheet 28 over the second adhesive 26 that can be removed from the device 10 when adhering the device 10 directly to the wearer's skin. When the second adhesive cover sheet 28 is removed and the device 10 is adhered directly to the wearer's skin, the first removable cover sheet 24 can remain in place to prevent objects, such as dirt, dust, or the wearer's hair, from accidently or unexpectedly adhering to the first adhesive 20. If the wearer does not wish to have adhesive directly contact his or her skin, when the device 10 is adhered to the underside of the wearable item, such as the head covering, the second removable cover sheet 28 can remain in place to separate the wearer's skin from the second adhesive 26.

To use the wearable indicator device 10, the wearer first removes either the first cover sheet 24 or the second cover sheet 28 from the wearable indicator device 10 to expose either the first adhesive 20 or the second adhesive 26. When the first removable cover sheet 24 is removed to expose the first adhesive 20, the wearer next presses the first surface 18 and the first adhesive 20 of the wearable indicator device 10 against a wearable item, such as a piece of clothing or head covering that is worn by the wearer. For example, the first surface 18 and adhesive 20 can be pressed against an underside of a hairnet, such that the adhesive 20 adheres to a fabric sheet or elastic strap of the hairnet.

Once the indicator device 10 is adhered to the hairnet, the wearer can place the hairnet on his or her head. The wearer then adjusts the hairnet so that the indictor device 10 is positioned over the wearer's forehead so that the temperature indicator 14 is visible to bystanders and so that the second surface 22 of the indicator device 10 and temperature indicator 14 are proximate to the wearer's skin. As previously described, the wearer can leave the second removable cover sheet 28 in place so that the second adhesive 26 does not contact and/or adhere to the wearer's skin.

Alternatively, the wearer can remove the second removable cover sheet 28 from the indicator device 10 to expose the second adhesive 26. The wearer then presses the second surface 22 and the second adhesive 26 against the skin to adhere the indicator device 10 directly to the wearer's skin. The wearer can leave the first cover sheet 24 attached to the device 10 to prevent foreign objects (i.e., dirt, dust, hair) from adhering to the adhesive 20 on the first surface 18 of the device 10.

Indicator Device Formed from Laminated Layers

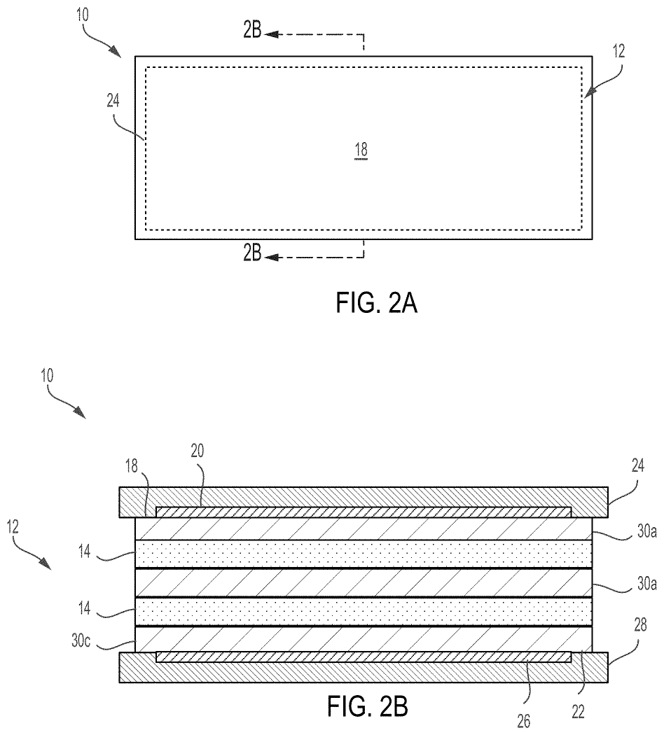

With reference to FIGS. 2A and 2B, another example of a wearable indicator device 10 is shown, which comprises a support structure 12 and visual temperature indicator 14. The support structure 12 and temperature indicator 14 are formed from polymeric layers of different materials that are laminated together to form the indicator device 10. The support structure 12 is a polymeric sheet having a first surface 18 and a second surface 22. The sheet comprises multiple layers 30a, 30b, 30c comprising polymeric materials, such as flexible polymeric materials comprising polyvinyl chloride, polyethylene, polyurethane, or latex. The wearable support structure 12 can also comprise the first adhesive 20 on the first surface 18, first cover sheet 24 over the adhesive 20, and, optionally, the second adhesive 26 on the second surface 22 of the support structure 12 and the second cover sheet 28 over the second adhesive 26.

The device 10 further comprises the visual temperature indicator 14. The indicator 14 is formed from one or more polymeric layers laminated between layers 30a, 30b, 30c of the support structure 12. The polymeric layers can be mixed or impregnated with one or more temperature sensitive materials that change color when exposed to selected temperatures and/or temperature changes. The layers 30a, 30b, 30c of the support structure 12 can be transparent or translucent so that color change of the layer(s) of the visual temperature indictor 14 are visible to bystanders and/or to the wearer of the indicator device 10.

Hairnet with Wearable Indicator Device

In the previously described examples, the wearable indicator device 10 is a separate disposable or reusable device that is configured to be adhered to a wearable item or directly to the patient's skin. In other examples, wearable indicator devices 110, 210 can comprise visual temperature indicators 114, 214 that are provided, integrally formed with, and/or permanently attached to wearable items, such as head coverings, garments, or bracelets. Permanently attaching the visual temperature indicators 114, 214 to the wearable items can provide a more secure connection between the indicators 114, 214 and the wearable item than is provided by the previously described adhesives 20, 26 and can ensure that the indicator 114, 214 is properly positioned and can be easily seen by bystanders every time that the wearable indicator device 10, 210 is used.

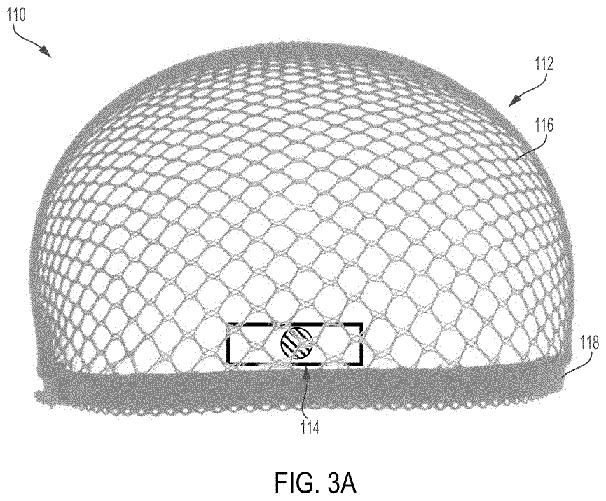

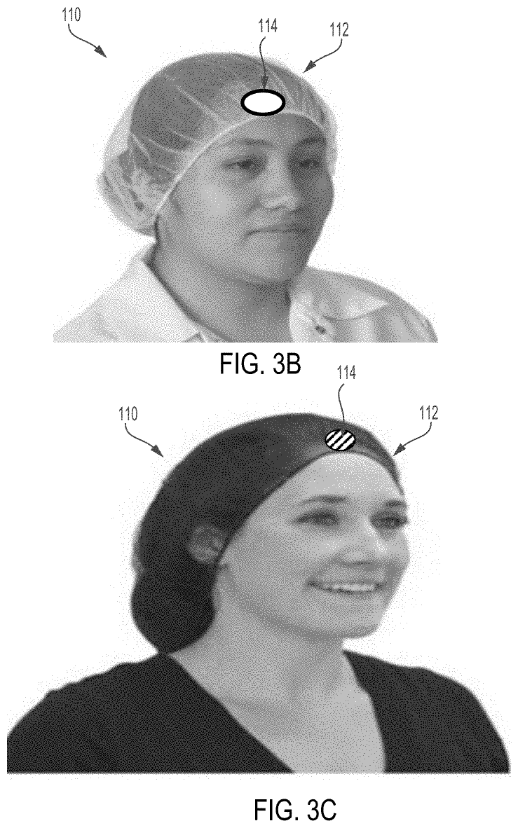

For example, with reference to FIGS. 3A-3C, a wearable indicator device 110 comprises a wearable support structure 112 in the form of a hairnet, such as a hairnet worn by cafeteria workers, and a visual temperature indictor 114. The support structure 112 or hairnet comprises a fabric sheet 116 that covers at least a portion of a wearer's head and/or hair. The fabric sheet 116 can be formed from any lightweight fabric, such as cotton or synthetic materials, such as Lycra, spandex, nylon, or polyester. The fabric sheet 116 can also comprise a mesh or knit sheet formed from threads that are twisted together to form a suitable head covering. The hairnet further comprises at least one elastic strap 118 connected to the fabric sheet positioned to secure the fabric sheet to the wearer's head.

The visual temperature indicator 114 is connected to the fabric sheet 116 and/or elastic strap 118 of the hairnet, and is positioned to be worn over the wearer's forehead, as shown in FIGS. 3B and 3C. The visual temperature indicator 114 can be similar to any of the temperature indicator(s) 14 described in previous examples. In particular, the visual temperature indicator 114 can include temperature sensitive materials embedded, for example, in a polymeric sheet or film. As shown in FIG. 3B, the visual temperature indicator 114 has a first appearance when the wearer has a normal body temperature. As shown in the figures, the first appearance is represented by a white indicator 114. However, this appearance is not intended to limit the scope of the present disclosure, and many different appearance options for the indicator 114 will be apparent to those skilled in the art. For example, the indicator 114 could be green to indicate that the wearer does not have a fever and is safe for bystanders to approach. The visual temperature indicator 114 changes to a second appearance when the wearer has a fever. As shown in the figures, the “second appearance” is represented when the indicator 114 is filled by a cross hatch pattern. In other examples, the indicator 114 may change to another color when a fever is detected. For example, the indicator 114 may be green when the wearer is safe (i.e., does not have a fever) and red when a fever is detected.

It is expected that the hairnet will be worn by cafeteria workers and other food service providers when performing work activities. The conspicuously positioned visual temperature indictor 114 provides a visual confirmation for customers and other employees that the wearer does not have a fever and, accordingly, is less likely to be infected with coronavirus or other infections. Beneficially, this visual confirmation is provided in substantially real-time, as the wearer performs his or her work activities and not only, for example, at the beginning of a shift or when the wearer arrives for work.

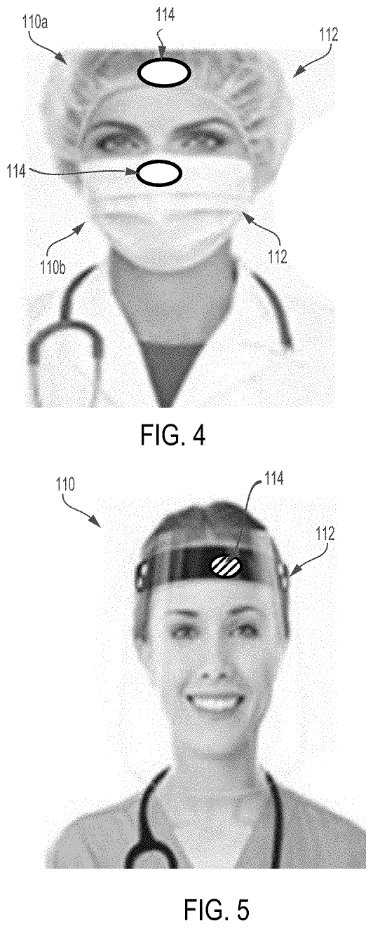

With reference to FIG. 4, a wearer, such as a medical provider (e.g., a physician, nurse, or medical technician) is wearing two wearable indicator devices (i.e., a first device 110a and a second device 110b). The first device 110a comprises a wearable structure 112 comprising a head covering. The visual temperature indicator 114 is secured to the elastic band of the head covering, as in previous examples. The second device 110b comprises a wearable structure 112 in the form of a facemask, covering the wearer's nose and mouth. The visual temperature indicator 114 is secured to the facemask, resting above the bridge of the wearer's nose.

FIG. 5 shows a wearer, such as a medical professional, wearing a wearable indicator device 110 comprising a support structure 112 in the form of a face shield comprising a plastic shield secured to the wearer's head by a headband. The visual temperature indicator 114 is secured to the headband portion of the face shield and is positioned at a conspicuous location on the wearer's forehead.

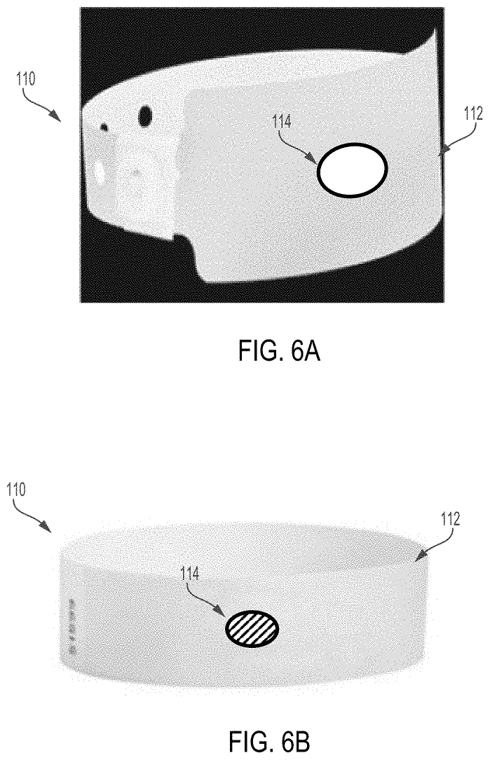

With reference to FIGS. 6A and 6B, the support structure 112 of the indicator device 110 comprises a disposable bracelet, such as a disposable bracelet provided to a patient upon arrival at an emergency treatment and/or urgent care facility. The visual temperature indicator 114 is connected to and/or extends through a portion of the bracelet. As shown in FIG. 6A, the visual temperature indicator 114 has the first appearance, showing that the wearer has a normal body temperature. In FIG. 6B, the visual temperature indicator 114 has the second appearance, showing that the wearer has a fever.

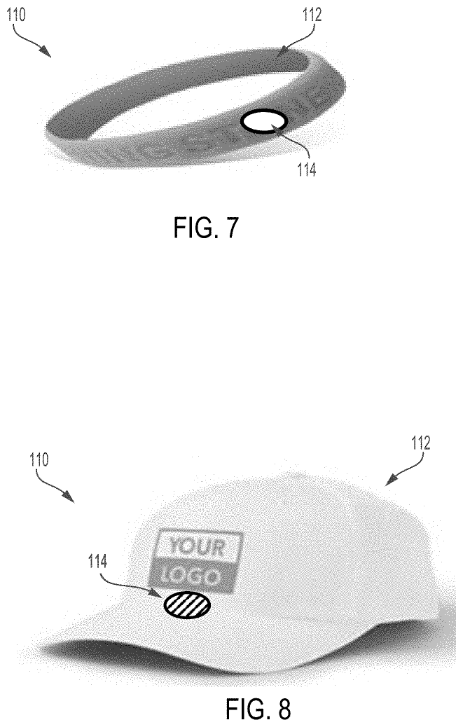

With reference to FIG. 7, the support structure 112 of the indicator device 110 also comprises a bracelet. Unlike in previous examples, the bracelet in FIG. 7 can be washable and reusable. For example, the bracelet can be formed from silicone, rubber, neoprene, or similar materials, which can be washed several or many times before needing to be thrown away. In some examples, the visual temperature indicator 114 can be integral or co-molded with the bracelet material forming a secure connection between the support structure 112 and visual temperature indicator 114. In other examples, the visual temperature indicator 114 can be connected to the support structure 112 after the support structure 112 is molded or formed using conventional connectors or adhesives, as previously described.

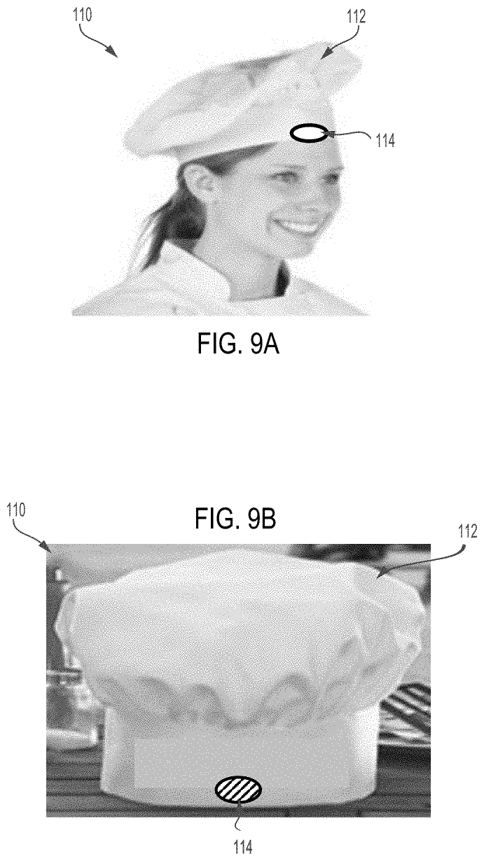

With reference to FIGS. 8, 9A, and 9B, the support structure 112 of the wearable indicator device 110 comprises a hat. For example, as shown in FIG. 8, the hat is a conventional baseball cap. The visual temperature indicator 114 can be secured to the cap portion of the hat to ensure that the visual temperature indicator 114 remains proximate to the wearer's skin (i.e., the wearer's forehead). The baseball cap may be worn, for example, by fans entering a stadium or arena. Stadium or arena employees, such as ticket-takers and turnstile attendants, may be trained to look for the visual temperature indicator 114 on fans' clothing to identify individuals entering the stadium or arena that have a fever. As shown in FIGS. 9A and 9B, the hat is a chef's hat. The hat may be worn by chefs while preparing food to provide visual confirmation for customers and other employees that the wearer does not have a fever.

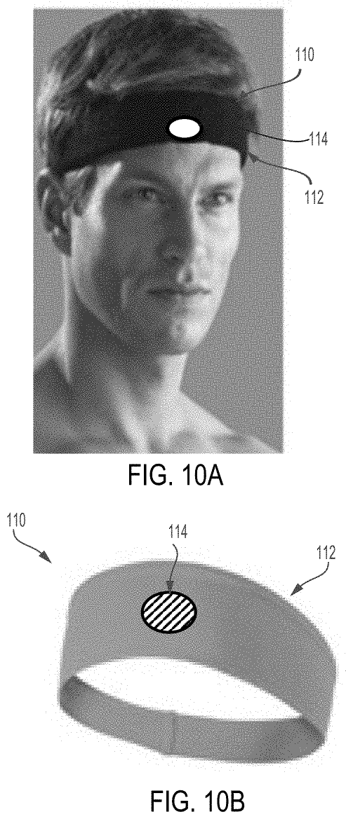

With reference to FIGS. 10A and 10B, the support structure 112 of the wearable indicator device 110 comprises a headband, such as a headband worn by individuals participating in exercises and other athletic activities. The visual temperature indicator 114 is connected to and/or extends through the headband. As shown in FIG. 10A, the visual temperature indicator 114 can be positioned over a central portion of the wearer's forehead so that it is easily visible to others.

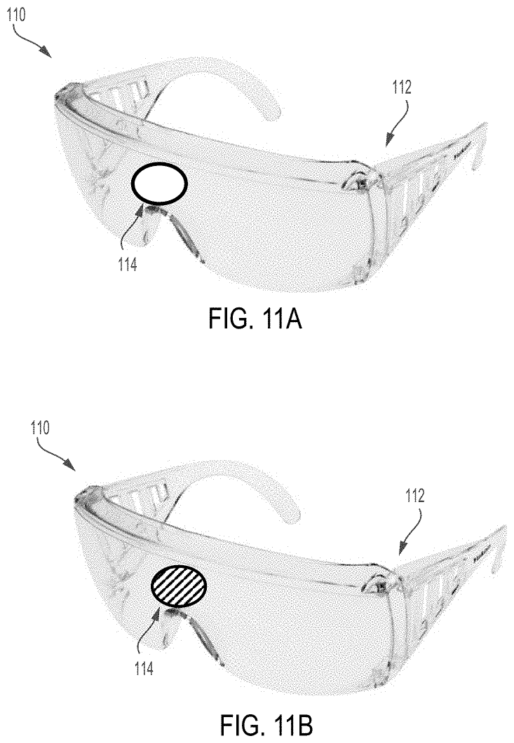

With reference to FIGS. 11A and 11B, the support structure 112 of the device 110 comprises safety glasses, such as safety glasses worn by individuals in a laboratory or medical facility. The visual temperature indictor 114 is connected to the safety glasses at a position above the nosepiece of the safety glasses. As shown in FIG. 11A, the visual temperature indicator 114 has the first appearance, showing that the wearer's body temperature is normal. The visual temperature indicator 114 in FIG. 10B has the second appearance indicating that the wearer has a fever.

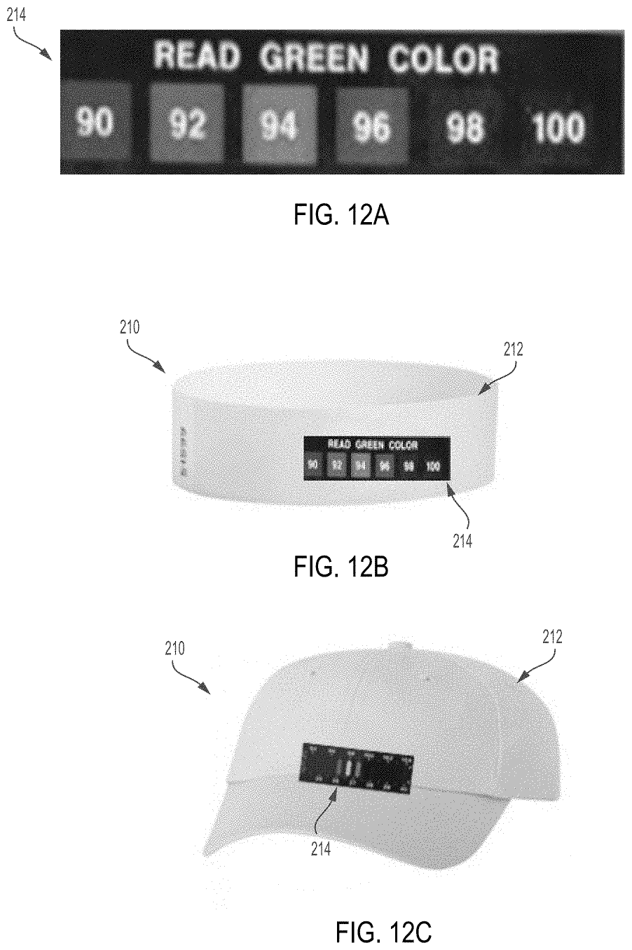

With reference to FIGS. 12A-12C, another example of a visual temperature indicator 214 for a wearable indicator device 210 is shown. The visual temperature indicator 214 can be connected or mounted to any of the previously described support structures 12, 112, such as to any of the previously described plastic sheets, head coverings, hats, headbands, and wrist bands. Unlike in previous examples, where the visual indicator 14, 114 indicated only whether the wearer had a normal body temperature or a fever, the visual temperature indicator 214 comprises multiple (e.g., two or more) portions representing different temperatures sensed by the indicator 214. The portions can be arranged in a strip, grid pattern, or any other convenient arrangement. The different portions change in appearance in response to different temperatures to provide a more detailed indication of the wearer's body temperature. Accordingly, the indicator 214 shows not only whether a wearer has a fever, but whether the wearer has an abnormally low body temperature and/or a severity of the fever. For example, by viewing the indicator 214, an observer can see whether the wearer has a body temperature of 90° F., 92° F., 94° F., 96° F., 98° F., or 100° F. As discussed previously, the visual temperature indicator 214 can be connected to any of the previously described support structures. For example, as shown in FIG. 12B, the support structure 212 can be a wristband. As shown in FIG. 12C, the support structure 212 can be a hat.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements. Furthermore, it is to be understood that the present invention contemplates that, to the extent possible, one or more features of any embodiment can be combined with one or more features of any other embodiment.

The invention claimed is:

1. A wearable indicator device, comprising:
a flexible sheet comprising a first surface comprising an adhesive covering at least a portion of the first surface and a second surface configured to be positioned proximate to skin of a wearer of the wearable indicator device; and
a visual temperature indicator configured to be visible to bystander(s) when the wearable indicator device is being worn and which changes in appearance when exposed to a temperature corresponding to a temperature exceeding a normal body temperature of the wearer to provide a visual indication when the wearer has an abnormal body temperature,
wherein the visual temperature indicator is connected to, embedded in, or integral with the flexible sheet, such that the flexible sheet is configured to hold the visual temperature proximate to the skin of the wearer.

2. The indicator device of claim 1, wherein the visual temperature indicator comprises a temperature-sensitive material that undergoes a chemical reaction causing the material to change in appearance when exposed to the temperature corresponding to the temperature exceeding normal body temperature.

3. The indicator device of claim 2, wherein the temperature-sensitive material comprises at least one of a solution, film, coating, powder, or particles integral with or embedded in a polymeric substrate.

4. The indicator device of claim 2, wherein the temperature sensitive material has a first appearance when the temperature to which the temperature sensitive material is exposed corresponds to normal body temperature, and the temperature sensitive material has a second appearance when the temperature to which the temperature sensitive material is exposed corresponds to abnormal body temperature.

5. The indicator device of claim 4, wherein the temperature sensitive material is configured to transition between the first appearance and the second appearance multiple times as the temperature to which the temperature sensitive material is exposed changes between the temperature corresponding to normal body temperature and the temperature corresponding to abnormal body temperature.

6. The indicator device of claim 4, wherein the temperature sensitive material initially has the first appearance, transitions to the second appearance when the temperature to which the temperature sensitive material is exposed corresponds to abnormal body temperature, and is incapable of returning to the first appearance following the transition from the first appearance to the second appearance.

7. The indicator device of claim 4, wherein the temperature sensitive material has the first appearance when at least a portion of the temperature sensitive material that can be seen by the bystander(s) comprises a first color, and the temperature sensitive material has the second appearance when the portion of the temperature sensitive material changes from the first color to a different second color.

8. The indicator device of claim 7, wherein the temperature sensitive material has a third appearance when the temperature to which the temperature sensitive material is exposed corresponds to the wearer having a body temperature significantly exceeding normal body temperature by more than about 3 degrees Fahrenheit.

9. The indicator device of claim 1, wherein the visual temperature indicator changes in appearance when exposed to the temperature corresponding to the abnormal body temperature by displaying at least one of a word, phrase, or icon visible to the bystander(s) indicating to the bystander(s) that at least one of (i) the wearer has a fever, (ii) the wearer should seek medical attention, or (iii) the bystander(s) should remain socially distanced from the wearer.

10. The indicator device of claim 1, wherein the visual temperature indicator has an inactive or pre-use appearance when exposed to temperatures significantly below normal body temperature to indicate that the visual temperature indicator is not in-use or is not correctly positioned proximate to the wearer's skin.

11. The indicator device of claim 1, wherein the flexible sheet comprising multiple laminated layers and the visual temperature indicator comprises at least one temperature-sensitive layer laminated within the multiple layers of the wearable support structure and at least partially visible through the multiple layers of the wearable support structure.

12. The indicator device of claim 11, wherein the multiple laminated layers comprise flexible polymer layers comprising at least one of polyvinyl chloride, polyethylene, polyurethane, or latex.

13. The indicator device of claim 1, wherein the visual temperature indicator comprises multiple portions configured to change color when exposed to different temperatures to provide an indication of a severity of the wearer's fever.

14. The indicator device of claim 1, wherein the first surface of the flexible sheet is configured to be adhered to an underside of a head covering worn by the wearer, the indicator device further comprising a removable cover sheet over the adhesive configured to be removed from the device prior to adhering the device to the underside of the head covering.

15. The indicator device of claim 1, wherein the first surface of the flexible sheet is configured to be adhered to an underside of a head covering worn by the wearer, the indicator device further comprising:
a second adhesive covering at least a portion of the second surface of the flexible sheet so that the device can be adhered either to the underside of the head covering or directly to the wearer's skin;
a first removable cover sheet over the adhesive on the first surface configured to be removed from the device when adhering the device to the underside of the head covering; and
a second removable cover sheet over the second adhesive on the second surface configured to be removed from the device when adhering the device directly to the wearer's skin.

16. A hairnet comprising:
a wearable support structure comprising a fabric sheet configured to cover at least a portion of a wearer's head and/or hair and at least one elastic strap connected to the fabric sheet positioned to secure the fabric sheet to the wearer's head; and
a visual temperature indicator mounted to the wearable support structure configured to be visible to bystander(s) when the hairnet is being worn and which changes in appearance when exposed to a temperature corresponding to a temperature exceeding a normal body temperature of the wearer to provide a visual indication when the wearer has an abnormal body temperature,
wherein the wearable support structure is configured to hold the visual temperature indicator proximate to a forehead of the wearer.

17. The hairnet of claim 16, further comprising a clip that attaches the visual temperature indicator to the wearable support structure.

18. The hairnet of claim 16, wherein the visual temperature indicator is removably connected to the wearable support structure by an adhesive.

19. A method of assembling a hairnet, the method comprising:
providing the hairnet comprising a fabric sheet configured to cover at least a portion of a wearer's head and/or hair and at least one elastic strap positioned to secure the fabric sheet to the wearer's head; and
adhering the first surface of the wearable support structure of the wearable indicator device of claim 1 to the fabric sheet of the hairnet by pressing the indicator device against an underside of the fabric sheet such that the adhesive adheres to the fabric sheet of the hairnet,
wherein, when the indicator device is adhered to the fabric sheet of the hairnet, the visual temperature indicator of the indicator device is positioned such that, when the hairnet is worn by the wearer, the visual temperature indicator is proximate to a forehead of the wearer.

20. The method of claim 19, wherein adhering the first surface of the wearable support structure to the fabric sheet comprises removing a cover sheet from the wearable indicator device to expose the adhesive and pressing the first surface and the adhesive of the wearable indicator device against the underside of the fabric sheet of the hairnet.
